(19) United States
(12) Patent Application Publication
(10) Pub. No.: US 2015/0368172 A1
(43) Pub. Date: Dec. 24, 2015

(54) Method for Producing Alpha, Beta-Unsaturated Alcohol

(71) Applicant: JX NIPPON OIL & ENERGY CORPORATION, Chiyoda-ku, Tokyo (JP)

(72) Inventors: Atsushi SEGAWA, Tokyo (JP); Daisuke WATANABE, Tokyo (JP); Junji WAKABAYASHI, Tokyo (JP)

(21) Appl. No.: 14/766,473

(22) PCT Filed: Feb. 6, 2014
(86) PCT No.: PCT/JP2014/053390

§ 371 Date: Aug. 7, 2015

(30) Foreign Application Priority Data
Feb. 8, 2013 (JP) 2013-023474

(51) Int. Cl. C07C 29/141

(52) U.S. Cl. CPC C07C 29/141

(57) ABSTRACT

A method of producing an α,β-unsaturated alcohol at a high conversion ratio and a high selectivity is provided, which comprises continuously supplying a gas mixture containing an α,β-unsaturated aldehyde and at least an equimolar amount of a secondary alcohol using zirconium oxide as a catalyst to thereby produce the corresponding α,β-unsaturated alcohol through a hydrogen transfer reaction from the secondary alcohol. According to this method, crotyl alcohol can be produced at a high yield from crotonaldehyde using isopropanol as a hydrogen source, the crotonaldehyde being obtained by dehydrogenating bioethanol to produce acetaldehyde and then subjecting acetaldehyde to aldol condensation.

### TECHNICAL FIELD

The present invention relates to a method of producing an α,β-unsaturated alcohol through a hydrogen transfer reaction using an α,β-unsaturated aldehyde as a raw material in the presence of a catalyst composed of zirconium. oxide. The present invention also relates to a method of producing an α,β-unsaturated alcohol, the method being characterized in that a secondary alcohol is used as a hydrogen donor in a hydrogen transfer reaction and that a ketone produced as a result of the hydrogen transfer reaction from the secondary alcohol is reduced with hydrogen to regenerate the secondary alcohol and this secondary alcohol is reused as the hydrogen donor. The present invention also relates to a method of producing an α,β-unsaturated alcohol, the method being characterized in that the production process includes a production step of an aliphatic aldehyde, which corresponds to a saturated aliphatic alcohol, by a dehydrogenation reaction of the saturated aliphatic alcohol and that the hydrogen obtained through the dehydrogenation reaction is used for the reduction reaction of the ketone.

### BACKGROUND ART

The α,β-unsaturated aldehyde, which is a raw material in the present invention, has a carbon-carbon double bond between the α position and β position in its molecule and has a carbonyl group containing a carbon atom adjacent to the alpha position and opposite to the β position in the molecule. In the production of the α,β-unsaturated alcohol using the α,β-unsaturated aldehyde as a raw material, the catalyst and the hydrogen donating compound must be appropriately designed and selected to increase the regioselectivity of the hydrogen transfer reaction. Otherwise, a problem occurs in that a byproduct and a heavy material (for example, a sequential aldol condensation reaction product) are produced.

α,β-Unsaturated alcohols are produced as solvents and raw materials for basic chemicals and have a variety of applications, so that expectations for novel methods of producing α,β-unsaturated alcohols are high. Particularly, expectations for a novel method of producing crotyl alcohol, which can be a raw material of butadiene that is estimated to be depleted in the future, are high. From the viewpoint of the requirements for a reduction in environmental load, expectations for the development of a method of producing an α,β-unsaturated alcohol using, as a starting material, ethanol obtained from a biomass resource are high.

Patent Literature 1 reports that, in a method using an alcohol as a hydrogen donator in the presence of a catalyst composed of an oxide of an element selected from the group consisting of yttrium, lanthanum, praseodymium, neodymium, and samarium, the conversion ratio of acrolein is about 68% at a reaction temperature of 340□C and the selectivity of allyl alcohol is about 85%.

Reactions for preparing allyl alcohol from acrolein using a catalyst and secondary butanol as a hydrogen donating source have been reported as follows: Patent Literature 2 reports a method using a catalyst containing yttrium and manganese oxides. Patent Literature 3 reports a method using a catalyst containing yttrium and cobalt oxides. Patent Literature 4 reports a method using a catalyst containing yttrium oxide as a main component and magnesium oxide as an accessory component. Patent Literature 5 reports a method using a catalyst containing cobalt oxide and an oxide of at least one element selected from lanthanum, cerium, praseodymium, neodymium, and samarium. Particularly, Patent Literature 3 reports that the conversion ratio of acrolein under the condition of 300° C. in the presence of the yttrium and cobalt oxide catalyst is about 64% at the maximum and the selectivity of allyl alcohol is about 85%.

In these Patent Literatures, although crotonaldehyde is exemplified as a raw material of the α,β-unsaturated aldehyde, there is no disclosure in Examples.

Patent Literature 6 discloses a catalyst for reducing a carbonyl compound to an alcohol and an improved method of converting an α,β-olefinic unsaturated aldehyde- or ketone-based compound to a corresponding allyl-based alcohol. This catalyst is composed mainly of tetragonal zirconium dioxide supported on a carrier. Patent Literature 6 reports that this method is performed in the presence of a supported tetragonal zirconium oxide catalyst or a supported catalyst selected from the group consisting of HfO₂, V₂O₅, NbO₅, etc. and that, when a ZrO₂/SiO₂ carrier catalyst is used, the conversion ratio of acrolein is 98% by weight and the yield of allyl alcohol is 87% by mole.

Patent Literature 7 discloses a method of producing an unsaturated alcohol. This method is characterized in that an unsaturated carbonyl compound is hydrogenated in an alcohol solvent under pressurization with molecular hydrogen in the presence of a catalyst composed of gold supported on an inorganic oxide carrier such as zirconia. Patent Literature 7 reports that, when acrolein is used as the raw material, allyl alcohol is produced at a conversion ratio of 49% and a selectivity of 74%.

Patent Literature 8 reports that crotyl alcohol is produced by hydrogenating crotonaldehyde under pressurization with molecular hydrogen using a hydrogenation reaction catalyst selective for carbonyl groups. This hydrogenation reaction catalyst is composed of a group VIII and period 6 element (iridium, osmium, or platinum) and a group IB element (gold, silver, or copper) that are in the form of ultrafine metal particles with an average particle diameter of 6 nm or less, and these particles are supported on a carrier such as zirconia. The crotyl alcohol is produced at a conversion ratio of 92% and a selectivity of 81%.

Patent Literature 9 discloses that an unsaturated alcohol is produced by hydrogenating a non-conjugated alicyclic unsaturated aldehyde in a vapor phase system in the presence of a catalyst composed of (A) copper alone or (B) copper and an oxide of a second metal selected from Cr, Fe, Al, Zn, etc. Patent Literature 9 reports that tetrahydrobenzaldehyde is used as a raw material and that an unsaturated alcohol corresponding to the raw material is produced through a hydrogenation reaction at 120° C. for 6 hours under pressurization with molecular hydrogen at a conversion ratio of 99.8% and a selectivity of 94%.

The catalysts used in the above Patent Literatures cannot be easily prepared because burning at high temperature for a long time is required or require a highly durable apparatus because of the conditions for the reaction, i.e., high temperature, high pressure, long time, etc. In addition, the conversion ratio and the selectivity may have a room for improvement in some cases. There is a disclosure that, in the synthesis of allyl alcohol using acrolein as a raw material, a hydrogen transfer reaction is utilized under pressurization with molecular hydrogen using 2-propanol as a solvent (Patent Literatures 1 to 5). However, there has been no disclosure of a reaction system in which crotyl alcohol is synthesized using crotonaldehyde as a raw material with isopropyl alcohol serving as a hydrogen donating source and the isopropyl alcohol is regenerated and reused, as in the present invention.

Non-Patent Literature 1 discloses that crotyl alcohol can be produced from crotonaldehyde in the presence of a zirconium oxide/silica catalyst using a secondary alcohol as a solvent in an autoclave filled with hydrogen at a pressure of 1 MPa (batch type). Non-Patent Literature 1 reports that the use of the Zr/SiO₂ carrier catalyst gives a conversion ratio of 99.4% and a selectivity of substantially 100%. However, there is no disclosure that the secondary alcohol is regenerated and reused in a continuous vapor phase method, as in the present invention.

### CITATION LIST

### Patent Literature

Patent Literature 1: Japanese Patent Application Laid-Open No. Hei. 6-56722

Patent Literature 2: Japanese Patent Application Laid-Open No. Hei. 7-109238

Patent Literature 3: Japanese Patent Application Laid-Open No. Hei. 7-109239

Patent Literature 4: Japanese Patent Application Laid-Open No. Hei. 7-204507

Patent Literature 5: Japanese Patent Application Laid-Open No. Hei. 7-204509

Patent Literature 6: Japanese Patent Application Laid-Open No. Hei. 6-226093

Patent Literature 7: Japanese Patent Application Laid-Open No. 2003-183201

Patent Literature 8: Japanese Patent Application Laid-Open No. 2003-284952

Patent Literature 9: Japanese Patent Application Laid-Open No. 2001-354607

### Non-Patent Literature

Non-Patent Literature 1: Abstracts of 84th catalyst forum, September, 1999 (“Catalysts and Catalysis,” September, 1999, Vol. 41, No. 6, pp 389 to 391)

### SUMMARY OF INVENTION

### Technical Problem

An object of the present invention is to provide an α,β-unsaturated alcohol production method that uses a catalyst that can be easily prepared and an easily available hydrogen donor to produce, from an α,β-unsaturated aldehyde, a corresponding α,β-unsaturated alcohol at a high conversion ratio and a high selectivity.

### Solution to Problem

A first aspect of the present invention relates to an α,β-unsaturated alcohol production method comprising continuously supplying a gas mixture containing an α,β-unsaturated aldehyde and at least an equimolar amount of a secondary alcohol in the presence of a catalyst containing zirconium oxide to thereby produce an α,β-unsaturated alcohol corresponding to the α,β-unsaturated aldehyde through a hydrogen transfer reaction from the secondary alcohol.

A second aspect of the present invention relates to an α,β-unsaturated alcohol production method comprising continuously supplying a gas mixture containing an α,β-unsaturated aldehyde and at least an equimolar amount of a secondary alcohol in the presence of a catalyst containing zirconium oxide to thereby produce an α,β-unsaturated alcohol corresponding to the α,β-unsaturated aldehyde through a hydrogen transfer reaction from the secondary alcohol, the production method being characterized by further comprising at least the following two steps:

(1) the step of hydrogenating a ketone produced from the secondary alcohol to regenerate the secondary alcohol; and

(2) the step of supplying the regenerated secondary alcohol to the hydrogen transfer reaction.

A third aspect of the present invention relates to an α,β-unsaturated alcohol production method comprising continuously supplying a gas mixture containing an α,β-unsaturated aldehyde and at least an equimolar amount of a secondary alcohol in the presence of a catalyst containing zirconium oxide to thereby produce an α,β-unsaturated alcohol corresponding to the α,β-unsaturated aldehyde through a hydrogen transfer reaction from the secondary alcohol, the production method being characterized by further comprising at least the following four steps:

(1) the step of hydrogenating a ketone produced from the secondary alcohol to regenerate the secondary alcohol;

(2) the step of supplying the regenerated secondary alcohol to the hydrogen transfer reaction;

(3) the step of dehydrogenating a saturated primary aliphatic alcohol having a carbon number equal to one half of the carbon number of the α,β-unsaturated alcohol to produce an aliphatic aldehyde corresponding to the saturated primary aliphatic alcohol; and

(4) the step of using, in the step (1), hydrogen obtained in the step (3).

A fourth aspect of the present invention relates to the α,β-unsaturated alcohol production method according to any one of the first to third aspects of the present invention, characterized in that the α,β-unsaturated aldehyde is crotonaldehyde, and the α,β-unsaturated alcohol is crotyl alcohol.

A fifth aspect of the present invention relates to the α,β-unsaturated alcohol production method according to any one of the first to fourth aspects of the present invention, characterized in that the secondary alcohol is isopropyl alcohol.

A sixth aspect of the present invention relates to the α,β-unsaturated alcohol production method according to any one of the first to fifth aspects of the present invention, characterized in that the gas mixture containing the α,β-unsaturated aldehyde and at least an equimolar amount of the secondary alcohol is supplied to a catalyst-packed column packed with zirconium oxide at LHSV, i.e. a liquid hourly space velocity of 0.1 to 20 h⁻¹.

### Advantageous Effects of Invention

According to the present invention, an α,β-unsaturated alcohol can be obtained at a high conversion ratio and a high selectivity from an α,β-unsaturated aldehyde using a catalyst that can be easily prepared. The above α,β-unsaturated aldehyde is obtained by dehydrogenating an alcohol to produce an aldehyde and then subjecting the obtained aldehyde to aldol condensation. Particularly, according to the present invention, crotyl alcohol can be produced using bioethanol as a raw material while hydrogen obtained through a dehydrogenation reaction of the bioethanol is effectively utilized.

### BRIEF DESCRIPTION OF DRAWINGS

FIG. 1 is an exemplary flow sheet for illustrating an embodiment of the present invention.

### DESCRIPTION OF EMBODIMENTS

### Catalyst and Catalyst Preparation

A main active component constituting a catalyst in the present invention is an oxide of zirconium. Specifically, the catalyst is a solid catalyst containing the zirconium oxide or a catalyst in which the zirconium oxide is supported on a carrier. The catalyst may be in any of a columnar form, a tablet form, a powdery form, and a granular form. From the viewpoint of efficient separation of the catalyst from the products and of high speed continuous treatment, it is preferable to use a granular catalyst which gives only a small pressure loss in a packed column packed with the catalyst and used for the reaction.

No particular limitation is imposed on the carrier so long as it can support the zirconium oxide. For example, a conventional catalyst support for a hydrogenation reaction can be used. Examples of such a carrier may include various carriers such as various metal oxides, zeolite, mesoporous silicates, and activated carbon. Of these, oxides and composite oxides are preferred. Specifically, silica, alumina, titania, zirconia, magnesia, silica-alumina, titania-zirconia, silica-magnesia, etc. are preferably used.

No particular limitation is imposed on the method of producing the catalyst, and the catalyst may be produced by any known method such as an impregnation method, a precipitation method, or a coprecipitation method, so long as the zirconium oxide serving as the active component is sufficiently dispersed in a final form. Any method or step may be used to cause the catalyst carrier to contain the active component or to be impregnated with the active component, so long as the activity of the zirconium oxide is not substantially inhibited. Examples of the catalyst production method may include: an impregnation method including impregnating pre-molded porous carrier grains or fine particles with a precursor of the active component soluble in water, alcohol, or a solvent and then performing drying and burning; and a precipitation method in which the catalyst is prepared from an aqueous solution of a salt of the active component by precipitation. Of these, a method including impregnating a mesoporous silicate with a zirconium-based compound and subjecting the resultant mesoporous silicate to burning treatment is preferred.

### (Catalyst-Packed Column and LHSV (Liquid Hourly Space Velocity))

In the present invention, the catalyst containing the zirconium oxide is packed into, for example, a packed column and fixed therein before use. No particular limitation is imposed on the shape, diameter, and height of the catalyst-packed column and the method of packing the catalyst.

As can be seen from the results in Examples described later under the condition of 200° C., there is a preferred range for the time of contact between the α,β-unsaturated aldehyde and the catalyst. The reaction may not proceed sufficiently when the time of contact is outside this range. Generally, LHSV (1/hr) is within the range of preferably 0.1 to 20 and more preferably 0.3 to 15.

### (α,β-Unsaturated Aldehyde)

In the present invention, hydrogen is donated from the secondary alcohol to the α,β-unsaturated aldehyde, and the α,β-unsaturated aldehyde is subjected to selective hydrogenation, whereby an α,β-unsaturated alcohol corresponding to the α,β-unsaturated aldehyde is produced. Examples of the α,β-unsaturated aldehyde used in the present invention include acrolein, methacrolein, crotonaldehyde, cinnamaldehyde, and tiglic aldehyde. Of these, crotonaldehyde is preferred because bioethanol can be used as a raw material and crotyl alcohol that can be used as a raw material of highly demanded butadiene is obtained.

### (Secondary Alcohol)

In the present invention, a secondary alcohol is used as a hydrogen donor. The secondary alcohol becomes a ketone after donation of hydrogen. Preferred examples of the secondary alcohol include isopropyl alcohol, 2-butanol, and 2-amyl alcohol. Of these, isopropyl alcohol is preferred because it has a boiling point lower than that of a corresponding α,β-unsaturated aldehyde and can easily become a vapor phase state and because the same applies to acetone, which is a ketone produced from isopropyl alcohol. The ratio of the secondary alcohol used to the α,β-unsaturated aldehyde is at least an equimolar ratio and preferably 2 to 30 in molar ratio.

### (Process Flow Sheet)

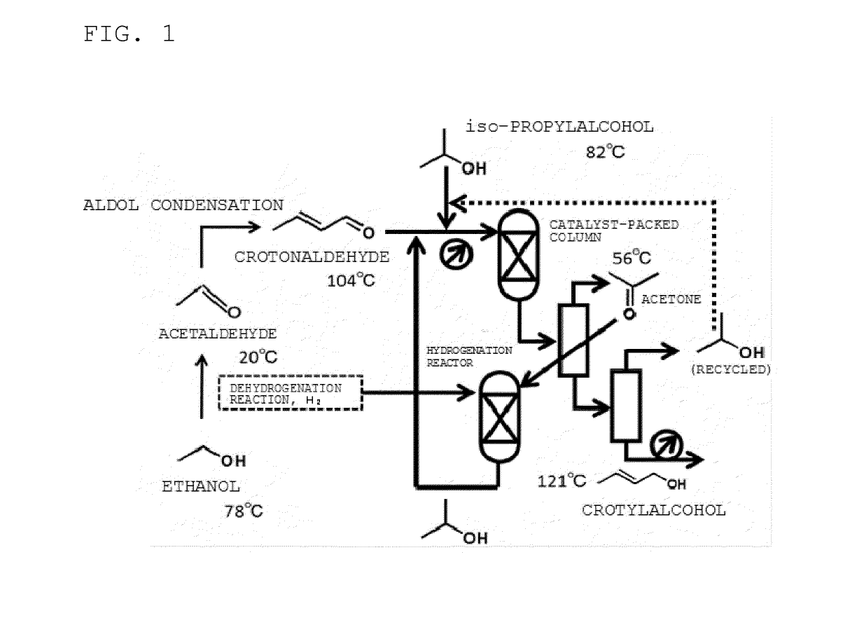

FIG. 1 shows an exemplary flow sheet for illustrating the embodiment of the present invention. In the description of this example, the α,β-unsaturated aldehyde is crotonaldehyde, and the secondary alcohol is isopropyl alcohol.

The flow sheet in FIG. 1 shows an example of the production of crotyl alcohol as the α,β-unsaturated alcohol using crotonaldehyde as the α,β-unsaturated aldehyde. In this example, the hydrogen source used is ethanol that can be obtained from a biomass resource and is a primary alcohol having a carbon number of 2, which is one half of the carbon number of crotyl alcohol, and hydrogen obtained by a dehydrogenation reaction of ethanol is used. The production of an acetaldehyde through a dehydrogenation reaction of ethanol is well-known, and a reaction for obtaining crotonaldehyde by aldol condensation of two acetaldehyde molecules is also well-known.

The crotonaldehyde is mixed with at least an equimolar amount of isopropyl alcohol, and the mixture is preheated and supplied to the catalyst-packed column. Then acetone produced from the isopropyl alcohol used for hydrogen donation is removed in a distillation column. This acetone is supplied to a hydrogenation reactor to regenerate isopropyl alcohol, and the regenerated isopropyl alcohol is used as a hydrogen donor to be mixed with crotonaldehyde. The residues in an acetone separation column are transferred to another distillation column, and unreacted (excess) isopropyl alcohol is removed. This isopropyl alcohol is used as the hydrogen donor for crotonaldehyde without any treatment. The residue is high-purity crotyl alcohol. If necessary, to further increase the purity, the high-purity crotyl alcohol may be subjected to a distillation column for purification, and the purified crotyl alcohol may be provided as a product.

### EXAMPLES

The present invention will next be further specifically described by way of Examples. The present invention is not limited to the scope described in the Examples so long as the present invention is defined by the scope of the claims. In FIG. 1, in order to show that the compounds in the flow sheet can be easily separated and can be brought into a vapor phase state, the boiling points of the compounds under normal pressure are shown.

### (Preparation of Catalyst)

An aqueous solution was prepared by dissolving 3.0 g of zirconium nitrate dihydrate (ZrO(NO₃)₂-2H₂O, manufactured by Wako Pure Chemical Industries, Ltd., molecular weight: 267.26) in 28 mL of water and was mixed with 20 g of spherical silica Q-10 (about 2 mm φ) manufactured by Fuji Silysia Chemical Ltd. to cause the zirconium to be supported on the silica. The obtained zirconium-supporting silica was dried overnight at 110° C. and then burned in air at 500° C. for 5 hours to obtain a zirconium oxide-supporting silica catalyst. The amount of the supported zirconium was about 5% by mass.

(Preparation of Catalyst in Comparative Examples) 7.50 g of aluminum nitrate nonahydrate (Al(NO₃)₃-9H₂O, manufactured by Wako Pure Chemical Industries, Ltd., molecular weight: 375.13) and 15.38 g of magnesium nitrate hexahydrate (Mg(NO₃)₂-6H₂O, manufactured by Wako Pure Chemical Industries, Ltd., molecular weight: 256.41) were simultaneously dissolved in water to prepare 100 mL of an aqueous solution (solution A). Separately, 200 mL of an aqueous solution (solution B) of 77.25 g of sodium carbonate decahydrate (Na₂CO₃-10H₂O, manufactured by Wako Pure Chemical Industries, Ltd., molecular weight: 286.14) was prepared. Then the solution A was added dropwise to the solution B at 60° C. to form precipitation. In this case, the precipitation was obtained while pH was maintained at 10 using a 1 mol/L aqueous sodium hydroxide solution. The obtained solution containing the precipitation was stirred at 80° C. for 24 hours, filtrated, and washed with pure water. To remove nitric acid ions contained in the precipitation, reflux washing was performed in an aqueous sodium carbonate solution (Na₂CO₃-10H₂O 0.64 g/water 300 mL), and then the resultant precipitation was again washed with pure water. The obtained precipitation was dried at 100° C. overnight to obtain 4.88 g of a solid. The solid was subjected to XRD measurement, and it was confirmed that the results agreed with those of Mg—Al-based hydrotalcite. Burning was performed at 500° C. in a nitrogen atmosphere for 8 hours, and the preparation of the catalyst was completed.

The results of the evaluation of this catalyst are shown under Comparative Example 3 in TABLE 1. Although the conversion ratio of crotonaldehyde was high, 90%, the selectivity of the target crotyl alcohol was very low, only 1%.

### (Production of α,β-Unsaturated Alcohol)

A raw material solution containing crotonaldehyde and isopropyl alcohol mixed at a prescribed molar ratio was continuously supplied to a SUS-made reaction tube (inner diameter: about 10 mm) packed with 6 mL of one of the above-obtained catalysts using a liquid pump at a prescribed LHSV (based on the sum of crotonaldehyde and isopropyl alcohol). A vaporizer was provided upstream of the reaction tube, and temperature was set to 120 to 150° C., whereby the raw material solution was gasified before introduction into the reaction tube. The reaction was performed at atmospheric pressure and a prescribed reaction temperature, and the reaction product was analyzed by gas chromatography. The results are shown in TABLE 1.

The conversion ratio (%) of crotonaldehyde and the selectivity (%) of crotyl alcohol were determined using the following formulas.

Conversion ratio of crotonaldehyde={1−(remaining crotonaldehyde/crotonaldehyde in raw material)}×100

Selectivity of crotyl alcohol=(yield of crotyl alcohol/conversion ratio of crotonaldehyde)×100

(Yield of crotyl alcohol=(crotyl alcohol produced/crotonaldehyde in raw material)×100)

TABLE 1

| | | Example 1 | Example 2 | Example 3 | Example 4 | Example 5 | Example 6 |
|---|---|---|---|---|---|---|---|
| Raw Materials | α,β-Unsaturated Aldehyde(A) | Crotonaldehyde | | | | | |
| | sec-Alcohol(B) | iso-Propylalcohol | | | | | |
| | primary-Alcohol(B) | — | — | — | — | — | — |
| Molar Ratio | (B)/(A) | 22 | 22 | 11 | 11 | 5.5 | 5.5 |
| Reaction | Temperature (° C.) | 110 | 200 | 110 | 200 | 110 | 150 |
| Condition | LHSV (hr−1) | 2 | 14 | 2 | 14 | 2 | 0.7 |

| | | | | | | |
|---|---|---|---|---|---|---|
| Conversion Ratio (%) | 75 | 89 | 57 | 78 | 17 | 96 |
| Selectivity (%) | 95 | 84 | 94 | 89 | 68 | 90 |

| | | | | Comparative | Comparative | Comparative |
|---|---|---|---|---|---|---|
| | | Example 7 | Example 8 | Example 1 | Example 2 | Example 3 |
| Raw Materials | α,β-Unsaturated Aldehyde(A) | Crotonaldehyde | | | | |
| | sec-Alcohol(B) | iso-Propylalcohol | | — | — | iso- |
| | | | | | | Propylalcohol |
| | primary-Alcohol(B) | — | — | Ethanol | | — |
| Molar Ratio | (B)/(A) | 5.5 | 2 | 22 | 22 | 22 |
| Reaction | Temperature (° C.) | 200 | 150 | 110 | 200 | 110 |
| Condition | LHSV (hr−1) | 14 | 0.4 | 2 | 14 | 2 |

| | | | | | |
|---|---|---|---|---|---|
| Conversion Ratio (%) | 53 | 90 | 17 | 43 | 90 |
| Selectivity (%) | 86 | 88 | 35 | 63 | 1 |

As can be seen from the results in TABLE 1, by reacting isopropyl alcohol with crotonaldehyde in the presence of a catalyst containing zirconium oxide, a hydrogen donating reaction proceeds, whereby the target crotyl alcohol can be obtained at high yield and high selectivity.

### INDUSTRIAL APPLICABILITY

The α,β-unsaturated alcohol containing crotyl alcohol and produced by the method of the present invention is used as a basic chemical such as a solvent without any treatment and is widely used as raw materials of specialty chemicals such as medical and agrochemical intermediates, perfumes, and industrial intermediates by utilizing the reactivity of the unsaturated bond.

1. An α,β-unsaturated alcohol production method comprising continuously supplying a gas mixture containing an α,β-unsaturated aldehyde and at least an equimolar amount of a secondary alcohol using zirconium oxide as a catalyst to thereby produce an α,β-unsaturated alcohol corresponding to the α,β-unsaturated aldehyde through a hydrogen transfer reaction from the secondary alcohol.

2. An α,β-unsaturated alcohol production method comprising continuously supplying a gas mixture containing an α,β-unsaturated aldehyde and at least an equimolar amount of a secondary alcohol using zirconium oxide as a catalyst to thereby produce an α,β-unsaturated alcohol corresponding to the α,β-unsaturated aldehyde through a hydrogen transfer reaction from the secondary alcohol, the production method being characterized by further comprising at least the following two steps:
(1) the step of hydrogenating a ketone produced from the secondary alcohol to regenerate the secondary alcohol; and
(2) the step of supplying the regenerated secondary alcohol to the hydrogen transfer reaction.

3. An α,β-unsaturated alcohol production method comprising continuously supplying a gas mixture containing an α,β-unsaturated aldehyde and at least an equimolar amount of a secondary alcohol using zirconium oxide as a catalyst to thereby produce an α,β-unsaturated alcohol corresponding to the α,β-unsaturated aldehyde through a hydrogen transfer reaction from the secondary alcohol, the production method being characterized by further comprising at least the following four steps:
(1) the step of hydrogenating a ketone produced from the secondary alcohol to regenerate the secondary alcohol;
(2) the step of supplying the regenerated secondary alcohol to the hydrogen transfer reaction;
(3) the step of dehydrogenating a saturated primary aliphatic alcohol having a carbon number equal to one half of the carbon number of the α,β-unsaturated alcohol to produce an aliphatic aldehyde corresponding to the saturated primary aliphatic alcohol; and
(4) the step of using, in step (1), hydrogen obtained in step (3).

4. The α,β-unsaturated alcohol production method according to any one of claims 1 to 3, characterized in that the α,β-unsaturated aldehyde is crotonaldehyde, and the α,β-unsaturated alcohol is crotyl alcohol.

5. The α,β-unsaturated alcohol production method according to any one of claims 1 to 3, characterized in that the secondary alcohol is isopropyl alcohol.

6. The α,β-unsaturated alcohol production method according to any one of claims 1 to 3, characterized in that the gas mixture containing the α,β-unsaturated aldehyde and at least an equimolar amount of the secondary alcohol is supplied to a catalyst-packed column packed with the zirconium oxide at a liquid hourly space velocity LHSV of 0.1 to 20 h⁻¹.

7. The α,β-unsaturated alcohol production method according to claim 4, characterized in that the secondary alcohol is isopropyl alcohol.

8. The α,β-unsaturated alcohol production method according to claim 4, characterized in that the gas mixture containing the α,β-unsaturated aldehyde and at least an equimolar amount of the secondary alcohol is supplied to a catalyst-packed column packed with the zirconium oxide at a liquid hourly space velocity LHSV of 0.1 to 20 h⁻¹.

9. The α,β-unsaturated alcohol production method according to claim 5, characterized in that the gas mixture containing the α,β-unsaturated aldehyde and at least an equimolar amount of the secondary alcohol is supplied to a catalyst-packed column packed with the zirconium oxide at a liquid hourly space velocity LHSV of 0.1 to 20 h⁻¹.
